(19) United States
(12) Patent Application Publication
(10) Pub. No.: US 2017/0052161 A1
(43) Pub. Date: Feb. 23, 2017

(54) GAS SENSING MATERIAL FOR A GAS SENSOR DEVICE

(71) Applicant: INVENSENSE, INC., San Jose, CA (US)

(72) Inventor: Fang Liu, San Jose, CA (US)

(21) Appl. No.: 15/047,344

(22) Filed: Feb. 18, 2016

Related U.S. Application Data
(60) Provisional application No. 62/208,091, filed on Aug. 21, 2015.

(51) Int. Cl. G01N 33/00

(52) U.S. Cl. CPC G01N 33/0027

(57) ABSTRACT

Gas sensing material for a gas sensor device is presented herein. In an implementation, a method includes generating a tin dioxide material by adding metal chloride and metal acetate to a mixture comprising tin dioxide and ammonium hydroxide, generating a precipitate substance by adding an organic solvent to the tin dioxide material, generating a slurry mixture from the precipitate substance by performing a centrifugation process and by adding water to the precipitate substance, generating a dry powder material by heating the slurry mixture via a heat treating process, generating an ink material by suspending the dry powder material in a surfactant substance, and printing the ink material onto a gas sensor.

### CROSS-REFERENCE TO RELATED APPLICATION

The present application claims priority to U.S. Provisional Patent Application No. 62/207,307, filed Aug. 19, 2015, the content of which application is hereby incorporated herein by reference in its entirety.

### TECHNICAL FIELD

The subject disclosure relates generally to gas sensing material for a gas sensor device.

### BACKGROUND

Certain gas sensors rely on physical changes or chemical changes in a chemical sensing material while in the presence of a gas to determine concentration of the gas in a surrounding environment. Further, certain chemical sensing materials preferentially operate at a temperature above normal ambient or room temperatures. However, conventional chemical sensing material comprises a high sensing temperature. Moreover, corrosive material is often employed and/or maintained in conventional chemical sensing material (e.g., resulting in corrosion of a substrate of a gas sensor that includes the conventional chemical sensing material, etc.).

### SUMMARY

The following presents a simplified summary of the specification to provide a basic understanding of some aspects of the specification. This summary is not an extensive overview of the specification. It is intended to neither identify key or critical elements of the specification nor delineate any scope particular to any embodiments of the specification, or any scope of the claims. Its sole purpose is to present some concepts of the specification in a simplified form as a prelude to the more detailed description that is presented later.

In accordance with an implementation, a method provides for generating a tin dioxide material by adding metal chloride and metal acetate to a mixture comprising tin dioxide and ammonium hydroxide, generating a precipitate substance by adding an organic solvent to the tin dioxide material, generating a slurry mixture from the precipitate substance by performing a centrifugation process and by adding water to the precipitate substance, generating a dry powder material by heating the slurry mixture via a heat treating process, generating an ink material by suspending the dry powder material in a surfactant substance, and printing the ink material onto a gas sensor.

In accordance with another implementation, a method provides for fabricating a tin dioxide material based at least on tin dioxide and a set of additives, removing corrosive material from the tin dioxide material during a fabrication process for fabricating an ink material based on the tin dioxide material, and printing the ink material on a pixel of a gas sensor device. The removing the corrosive material from the tin dioxide material can include performing a centrifugation process and performing a heat treating process.

In accordance with yet another implementation, a method provides for fabricating a tin dioxide material based at least on tin dioxide and a set of additives, removing corrosive material from the tin dioxide material during a fabrication process for fabricating an ink material based on the tin dioxide material, and printing the ink material on a pixel of a gas sensor device. The printing the ink material can include heating a substrate of the gas sensor device.

These and other embodiments are described in more detail below.

### BRIEF DESCRIPTION OF THE DRAWINGS

Various non-limiting embodiments are further described with reference to the accompanying drawings, in which:

FIG. 1 is a flowchart of an example methodology for fabricating ink material for a gas sensor, in accordance with various aspects and implementations described herein;

FIG. 2 depicts a gas sensor system for printing ink material, in accordance with various aspects and implementations described herein;

FIG. 3 depicts another gas sensor system for printing ink material, in accordance with various aspects and implementations described herein;

FIG. 4 depicts a gas sensor system for heating a gas sensor during printing of ink material, in accordance with various aspects and implementations described herein;

FIG. 5 depicts another gas sensor system for heating a gas sensor during printing of ink material, in accordance with various aspects and implementations described herein;

FIG. 6 is a flowchart of an example methodology for fabricating ink material, in accordance with various aspects and implementations described herein;

FIG. 7 is a flowchart of another example methodology for fabricating ink material, in accordance with various aspects and implementations described herein; and

FIG. 8 is a flowchart of yet another example methodology for fabricating ink material, in accordance with various aspects and implementations described herein.

### DETAILED DESCRIPTION

### Overview

While a brief overview is provided, certain aspects of the subject disclosure are described or depicted herein for the purposes of illustration and not limitation. Thus, variations of the disclosed embodiments as suggested by the disclosed apparatuses, systems, and methodologies are intended to be encompassed within the scope of the subject matter disclosed herein.

As described above, certain gas sensors rely on physical changes or chemical changes in a chemical sensing material while in the presence of a gas to determine concentration of the gas in a surrounding environment. Further, certain chemical sensing materials preferentially operate at a temperature above normal ambient or room temperatures. However, conventional chemical sensing material comprises a high sensing temperature. Moreover, corrosive material is often employed and/or maintained in conventional chemical sensing material (e.g., resulting in corrosion of a substrate of a gas sensor that includes the conventional chemical sensing material, etc.).

To these and/or related ends, various aspects and embodiments for fabricating ink material (e.g., gas sensing material, chemical sensing material, etc.) for a gas sensor are described. The various embodiments of the methods, techniques, and systems of the subject disclosure are described in the context of a gas sensor (e.g., a gas sensing device, a semiconductor gas sensor, a metal oxide semiconductor gas sensor, etc.) configured for sensing a gas in a surrounding environment. The novel ink material disclosed herein can be a nanoparticle based ink associated with chemical modification properties that can be employed to sense a gas. For example, the ink material can include nanoparticles (e.g., metal oxide nanoparticles) and organic material additives which can form an interconnected network of molecules (e.g., to improve porosity of the synthesized nanoparticles). In an aspect, independent metal(s) can be added to a metal oxide based gas sensor during a fabrication process disclosed herein. For example, independent metal(s) can be physically attached to a surface of the metal oxide. In another aspect, metal oxide nanoparticles can be generated during the fabrication process. As such, sensitivity, surface to volume ratio and/or performance of gas sensing material for the gas sensor can be improved. Furthermore, simplified manufacturability as compared to convention fabrication processes for fabricating gas sensing material can be provided. Moreover, corrosive material can be removed during the fabrication process disclosed herein (e.g., before applying the gas sensing material to the gas sensor). Therefore, damage to the gas sensor can be prevented. However, as further detailed below, various exemplary implementations can be applied to other areas of a gas sensing material and/or a gas sensor, without departing from the subject matter described herein.

### Exemplary Embodiments

Various aspects or features of the subject disclosure are described with reference to the drawings, wherein like reference numerals are used to refer to like elements throughout. In this specification, numerous specific details are set forth in order to provide a thorough understanding of the subject disclosure. It should be understood, however, that the certain aspects of disclosure may be practiced without these specific details, or with other methods, components, parameters, etc. In other instances, well-known structures and devices are shown in block diagram form to facilitate description and illustration of the various embodiments.

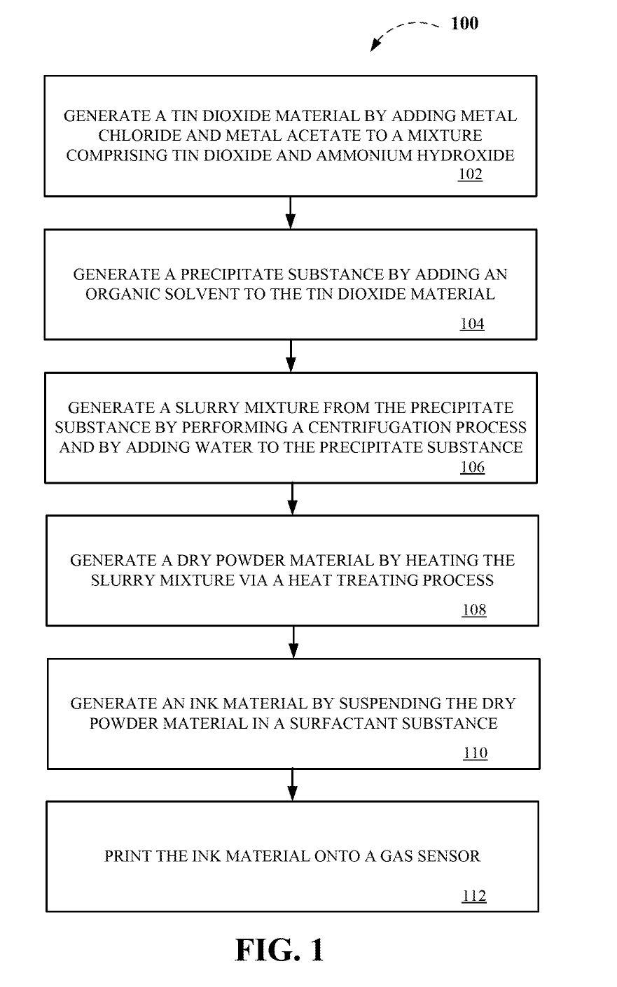

FIG. 1 illustrates an example, non-limiting embodiment of a method 100 for fabricating ink material (e.g., gas sensing material, chemical sensing material, etc.) for a gas sensor. The method 100 can be, for example, an ink synthesis method. In an aspect, the method 100 can be additionally employed for printing the ink material (e.g., the gas sensing material) on the gas sensor. Initially, at 102, a tin dioxide material is generated by adding metal chloride and metal acetate to a mixture comprising tin dioxide and ammonium hydroxide. For example, the mixture can comprise a tin dioxide powder and ammonium hydroxide. Therefore, a tin dioxide powder can be mixed with ammonium hydroxide, and metal chloride and/or metal acetate can then be added. Metal chloride is a metal salt containing chloride ion(s). Furthermore, metal acetate is a metal salt formed based on acetic acid. The metal chloride can comprise one or more types of metal. For example, the metal chloride can comprise platinum, lead, titanium, copper, zinc, lanthanide, iron, gold and/or another type of metal. Additionally or alternatively, the metal acetate can comprise one or more types of metal. For example, the metal acetate can comprise platinum, lead, titanium, copper, zinc, lanthanide, iron, gold and/or another type of metal. In an aspect, the tin dioxide material can be associated with metal nanoparticles and/or semiconductor nanoparticles. In another aspect, the tin dioxide material can comprise a tin dioxide core coated with material associated with the metal chloride and/or the metal acetate.

At 104, a precipitate substance is generated by adding an organic solvent to the tin dioxide material. For example, the precipitate substance can be generated by stirring the organic solvent with the tin dioxide material. The precipitate substance can comprise a solid form in response to the organic solvent. The precipitate substance comprises at least tin dioxide. In one example, the organic solvent can be ethanol. In another example, the organic solvent can be ethylene glycol. However, it is to be appreciated that the organic solvent can be a different type of organic solvent. Nanoparticles of the precipitate substance can be more porous than nanoparticles of the tin dioxide material due to the organic solvent.

At 106, a slurry mixture is generated from the precipitate substance by performing a centrifugation process and by adding water to the precipitate substance. For example, the slurry mixture can be generated from the precipitate substance by performing a centrifugation process and by adding deionized water to the precipitate substance. The slurry mixture can be a semiliquid mixture that comprises at least tin dioxide. Furthermore, the slurry mixture can be free of corrosive ions (e.g., corrosive chloride ions, corrosive hydroxide ions). For example, by performing the centrifugation process and by adding the water to the precipitate substance, chloride ions and/or hydroxide ions can be removed.

At 108, a dry powder material is generated by heating the slurry mixture via a heat treating process. The heat treating process can comprise heat treating the slurry mixture between 350° C. and 600° C. The dry powder material can be powder substance that comprises at least tin dioxide. Nanoparticles of the dry powder material can be in a solid form as a result of the heating.

At 110, an ink material is generated by suspending the dry powder material in a surfactant substance. The surfactant substance can lower surface tension of the dry powder material. For example, the surfactant substance can be a low-metal surfactant employed as a wetting agent to form the ink material. The ink material can be a printable ink. For example, the ink material can be a stable suspension ink for printing. In one example, the ink material can be a gas sensing material. The ink material comprises at least tin dioxide. As such, tin dioxide can be employed as a precursor to form the ink material.

At 112, the ink material is printed onto a gas sensor. The ink material can be printed on a gas sensor substrate. For example, the ink material can be printed on a microheater of a gas sensor (e.g., the ink material can be a gas sensing material and the gas sensing material can be printed on a microheater of gas sensor). In an aspect, ink material can be sintered at a temperature lower than 450° C. In another aspect, a substrate of the gas sensor can be heated to facilitate the printing of the ink material on the microheater of the gas sensor. For example, the substrate of the gas sensor can be heated via a metal layer that is heated between 20° C. and 450° C. to facilitate the printing of the ink material on the gas sensor.

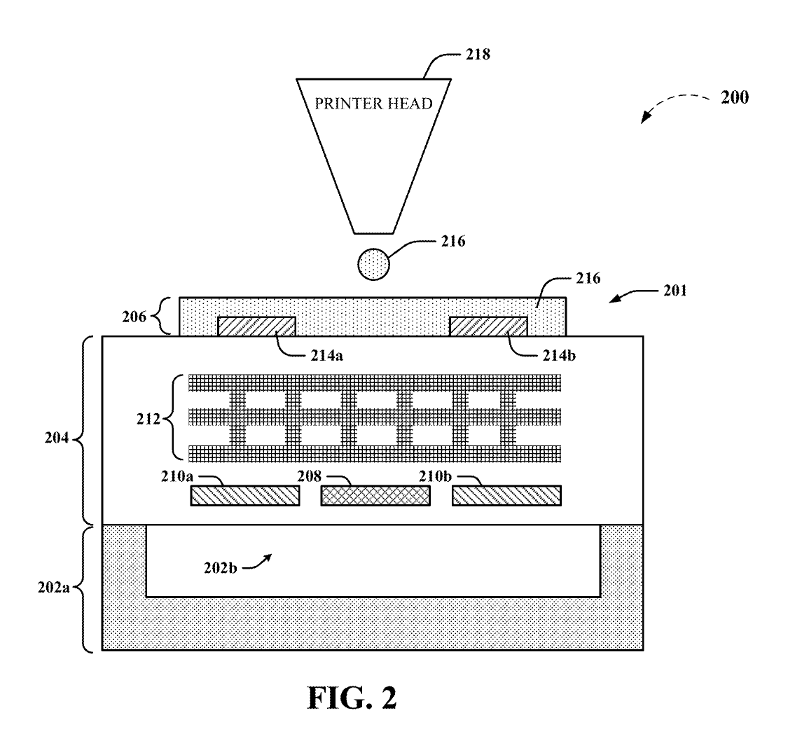

FIG. 2 depicts a system 200 that includes a gas sensor device 201, according to various non-limiting aspects of the subject disclosure. For example, the ink material mentioned above with respect to method 100 can be printed onto the gas sensor device 201. FIG. 2 depicts a cross-sectional view of the gas sensor device 201. The gas sensor device 201 can be, for example, a metal oxide semiconductor gas sensor. In an implementation, the gas sensor device 201 can include a CMOS substrate layer 202a, a dielectric layer 204 and a gas sensing layer 206. The dielectric layer 204 can be deposited or formed on the CMOS substrate layer 202a. For example, the dielectric layer 204 can be etched to the CMOS substrate layer 202a via wet etching or dry etching. Furthermore, the etching of the dielectric layer 204 to the CMOS substrate can be an isotropic etch or an anisotropic etch (e.g., a deep reactive ion etching, etc.). The CMOS substrate layer 202a can include a cavity 202b. The cavity 202b can thermally isolate the dielectric layer 204. In one example, the CMOS substrate layer 202a can be a gas sensor wafer. In another example, the CMOS substrate layer 202a can be a gas sensor die.

The dielectric layer 204 can provide mechanical support for temperature sensing elements and/or heating elements of the gas sensor device 201. The dielectric layer 204 can include a temperature sensor 208 and a heating element 210a-b. In one example, the heating element 210a-b can be implemented as a microheater. The temperature sensor 208 can be employed to sense temperature of the heating element 210a-b (e.g., the microheater). As such, the temperature sensor 208 and the heating element 210a-b can be implemented separate from the CMOS substrate layer 202a. In a non-limiting example, a thickness of the dielectric layer 204 can be approximately equal to 10 microns. However, it is to be appreciated that the dielectric layer 204 can comprise a different thickness.

The heating element 210a-b can include a first heating element 210a and a second heating element 210b. The temperature sensor 208 can be implemented between the first heating element 210a and the second heating element 210b in the same film deposition process. Film of the film deposition process can be, for example, polycrystalline silicon with different doping levels and/or metal silicide. Furthermore, the heating element 210a-b can be implemented as a micro-bridge structure. For example, the first heating element 210a can be configured as a first micro-bridge structure and the second heating element 210b can be configured as a second micro-bridge structure.

The temperature sensor 208 can be configured to sense temperature associated with the gas sensing layer 206. The temperature sensor 208 and the heating element 210a-b can be electrically and/or thermally coupled to a heat transfer layer 212. The heat transfer layer 212 can be associated with a set of metal interconnections (e.g., a set of metal vias). For example, the heat transfer layer 212 can comprise a set of metal interconnections that comprises aluminum, tungsten or another type of metal. Furthermore, the heat transfer layer 212 can include a plurality of metal layers that are electrically coupled via the set of metal interconnections. In an implementation, the temperature sensor 208, the heating element 210a-b and/or the heat transfer layer 212 can be suspended in the dielectric layer 204. For example, the temperature sensor 208, the heating element 210a-b and/or the heat transfer layer 212 can be surrounded by a dielectric material of the dielectric layer 204. The heat transfer layer 212 can transfer heat from a bottom portion of the dielectric layer 204 (e.g., a bottom portion of the dielectric layer 204 that is associated with the CMOS substrate layer 202a) to a top portion of the dielectric layer 204 (e.g., a top portion of the dielectric layer 204 that is associated with the gas sensing layer 206).

The gas sensing layer 206 can be deposited or formed on the dielectric layer 204. The gas sensing layer 206 can include a set of gas-sensing contacts 214a-b and an ink material 216. The ink material 216 can be a gas-sensing material (e.g., a chemical-sensing material). Furthermore, the ink material 216 can be fabricated via the method 100. For example, the ink material 216 can correspond to the ink material generated at step 110 of method 100. Therefore, the ink material 216 can be a printable ink (e.g., a stable suspension ink for printing). The ink material 216 can comprise at least tin dioxide. In an aspect, a printer head 218 can print the ink material 216 onto the gas sensor device 201 (e.g., onto a microheater of the gas sensor device 201, onto a pixel of the gas sensor device 201, onto the dielectric layer 204 of the gas sensor device 201, etc.). For example, the printer head 218 can be a printer head of a printer. Furthermore, the ink material 216 can be loaded into the printer head 218.

The gas-sensing contacts 214a-b can be electrically coupled to the ink material 216. In an aspect, the gas-sensing contacts 214a-b and at least a portion of the ink material 216 can be deposited or formed on the dielectric layer 204. The gas-sensing contacts 214a-b can be contact electrodes. The gas-sensing contacts 214a-b can be employed to detect changes in the ink material 216. For example, the gas-sensing contacts 214a-b can be employed to detect changes in the ink material 216 as a concentration of a target gas changes. The gas-sensing contacts 214a-b can be made of a conductive material, such as a noble metal. For example, the gas-sensing contacts 214a-b can comprise titanium nitride, poly-silicon, tungsten, another metal, etc. In one example, the gas-sensing contacts 214a-b can be electrically coupled to another component (e.g., an application-specific integrated circuit (ASIC)) of the gas sensor device 201.

The ink material 216 can be thermally coupled to the heating element 210a-b (e.g., the heating element 210a-b can provide heat to the ink material 216 of the gas sensing layer 206). For example, the dielectric layer 204 can provide thermal coupling between the heating element 210a-b and the ink material 216 so that the heat provided by the heating element 210a-b is conducted to the ink material 216. Accordingly, dielectric material of the dielectric layer 204 is preferably a low k dielectric material (e.g., a low k dielectric material relative to the CMOS substrate layer 202a and/or the gas sensing layer 206) with certain thermal conductivity. Furthermore, the ink material 216 can be exposed to an environment surrounding the gas sensor device 201. For illustration, the gas sensor device 201 can be associated with a sensor pixel (e.g., a single sensor pixel). For example, the ink material 216 can be configured to sense a type and/or a concentration of a certain gas. However, it is to be appreciated that the gas sensor device 201 can be configured with more than one sensor pixel that comprises one or more types of sensor pixels. Therefore, the gas sensor device 201 can be configured to detect numerous different gases at various concentrations. In a non-limiting example, the ink material 216 can be configured to sense carbon monoxide (CO) gas. In another non-limiting example, the ink material 216 can be configured to sense volatile organic compounds (VOC). However, it is to be appreciated that the ink material 216 can be configured to sense a different type of gas.

Furthermore, the ink material 216 can comprise a metal oxide having an electrical resistance based on a concentration of a gas in an environment surrounding the gas sensor device 201 and/or an operating temperature of the ink material 216. The ink material 216 can comprise an operating temperature greater than room temperature and determined by an amount of heat generated by the heating element 210a-b. The ink material 216 can comprise a metal oxide, such as but not limited to, an oxide of chromium, manganese, nickel, copper, tin, indium, tungsten, titanium, vanadium, iron, germanium, niobium, molybdenum, tantalum, lanthanum, cerium, neodymium or another type of metal. Alternatively, the ink material 216 can be composite oxides including binary, ternary, quaternary and complex metal oxides. The ink material 216 can be employed to detect chemical changes (e.g., chemical changes in response to a gas). For example, a conductivity change associated with the ink material 216 can be employed to detect a gas. In another example, a change of electrical resistance of the ink material 216 can be employed to detect a gas. In yet another example, a change of capacitance associated with the ink material 216 can be employed to detect a gas. However, it is to be appreciated that other changes associated with the ink material 216 (e.g., a change in work function, a change in mass, a change in optical characteristics, a change in reaction energy, etc.) can be additionally or alternatively employed to detect a gas.

In an implementation, a portion of the CMOS substrate layer 202a can be etched or otherwise removed to create the cavity 202b. The cavity 202b can be a thermal isolation cavity that thermally isolates the dielectric layer 204 and/or the gas sensing layer 206 from a bulk of the CMOS substrate layer 202a. The cavity 202b of the CMOS substrate layer 202a can allow integration of the dielectric layer 204 and/or the gas sensing layer 206 with other devices (e.g., an ASIC) and/or protects other devices from heat produced by the heating element 210a-b.

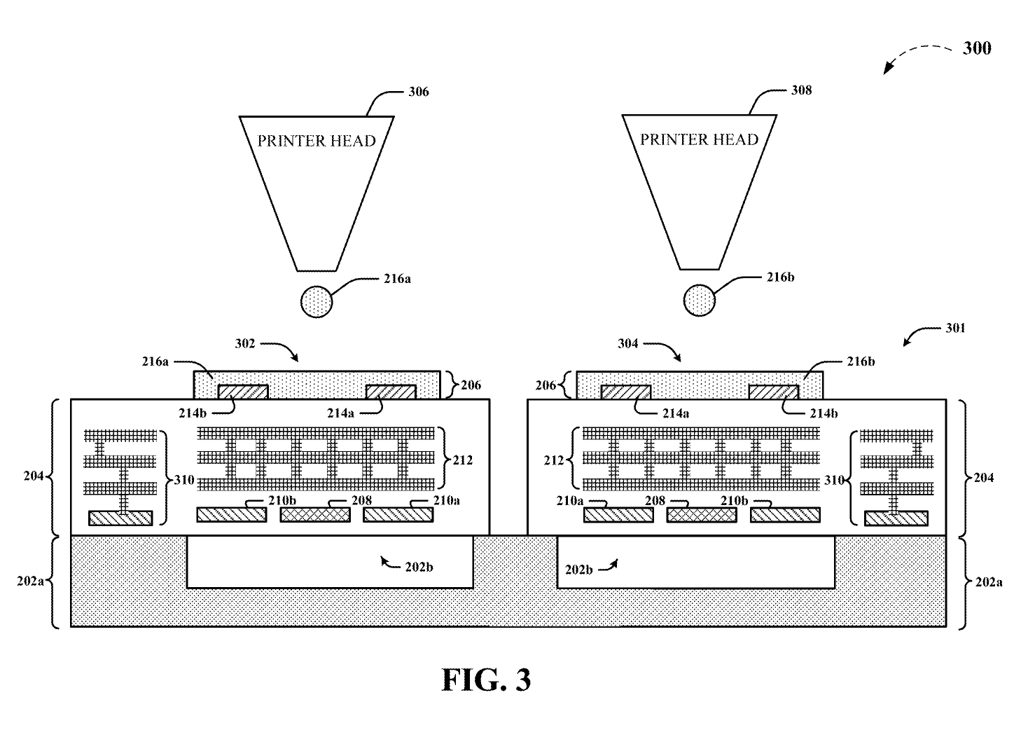

FIG. 3 depicts a system 300 that includes a gas sensor device 301, according to various non-limiting aspects of the subject disclosure. For example, the ink material mentioned above with respect to method 100 can be printed onto the gas sensor device 301. FIG. 3 depicts a cross-sectional view of the gas sensor device 301. The gas sensor device 301 can be, for example, a metal oxide semiconductor gas sensor. Furthermore, the gas sensor device 301 can be a multi-pixel gas sensing device. The gas sensor device 301 includes the CMOS substrate layer 202a, a first sensor pixel 302 and a second sensor pixel 304. In an implementation, the CMOS substrate layer 202a can include more than one cavity 202b (e.g., two cavities 202b).

The first sensor pixel 302 includes the dielectric layer 204 and the gas sensing layer 206. The dielectric layer 204 of the first sensor pixel 302 can include the temperature sensor 208, the heating element 210a-b and the heat transfer layer 212. Furthermore, the gas sensing layer 206 of the first sensor pixel 302 can include the set of gas-sensing contacts 214a-b and ink material 216a. The ink material 216a can be a gas-sensing material (e.g., a chemical-sensing material). Furthermore, the ink material 216a can be fabricated via the method 100. For example, the ink material 216a can correspond to the ink material generated at step 110 of method 100. Therefore, the ink material 216a can be a printable ink (e.g., a stable suspension ink for printing). The ink material 216a can comprise at least tin dioxide. For example, the ink material 216a can be fabricated via at least tin dioxide and a first set of additives. In an aspect, a printer head 306 can print the ink material 216a onto the gas sensor device 301 (e.g., onto a microheater of the gas sensor device 301, onto the first sensor pixel 302 of the gas sensor device 301, onto the dielectric layer 204 of the gas sensor device 301, etc.). For example, the printer head 306 can be a printer head of a printer. Furthermore, the ink material 216a can be loaded into the printer head 306.

Similarly, the second sensor pixel 304 includes the dielectric layer 204 and the gas sensing layer 206. The dielectric layer 204 of the second sensor pixel 304 can include the temperature sensor 208, the heating element 210a-b and the heat transfer layer 212. Furthermore, the gas sensing layer 206 of the second sensor pixel 304 can include the set of gas-sensing contacts 214a-b and ink material 216b. The ink material 216b can be a gas-sensing material (e.g., a chemical-sensing material). Furthermore, the ink material 216b can be fabricated via the method 100. For example, the ink material 216b can correspond to the ink material generated at step 110 of method 100. Therefore, the ink material 216b can be a printable ink (e.g., a stable suspension ink for printing). The ink material 216b can comprise at least tin dioxide. For example, the ink material 216b can be fabricated via at least tin dioxide and a second set of additives. In an aspect, a printer head 308 can print the ink material 216b onto the gas sensor device 301 (e.g., onto a microheater of the gas sensor device 301, onto the second sensor pixel 304 of the gas sensor device 301, onto the dielectric layer 204 of the gas sensor device 301, etc.). For example, the printer head 308 can be a printer head of a printer. Furthermore, the ink material 216b can be loaded into the printer head 308. In an implementation, the printer head 306 and the printer head 308 can be implemented in the same printer. In another implementation, the printer head 306 and the printer head 308 can be implemented in different printers.

In an implementation, the gas sensing layer 206 of the first sensor pixel 302 can be configured to sense a first type of gas and the gas sensing layer 206 of the second sensor pixel 304 can be configured to sense a second type of gas. For example, the ink material 216a can be employed to sense a first type of gas and the ink material 216b can be employed to sense a second type of gas. The first set of additives for the ink material 216a and the second set of additives for the ink material 216b can be associated with the metal chloride and/or the metal acetate from step 102 of the method 100. The first set of additives for the ink material 216a can determine the first type of gas sensed via the first sensor pixel 302. Furthermore, the second set of additives for the ink material 216b can determine the second type of gas sensed via the second sensor pixel 304. The metal chloride and/or the metal acetate associated with the first set of additives for the ink material 216a can be different than the metal chloride and/or the metal acetate associated with the second set of additives for the ink material 216b. For example, the first set of additives for the ink material 216a can comprise a different amount of metals (e.g., different amounts of platinum, lead, titanium, copper, zinc, lanthanide, iron, gold and/or other metal) than the second set of additives for the ink material 216b. However, it is to be appreciated that, in certain implementations, the gas sensing layer 206 of the first sensor pixel 302 and the gas sensing layer 206 of the second sensor pixel 304 can be configured to sense a corresponding type of gas.

Additionally, in certain implementation, the first sensor pixel 302 and the second sensor pixel 304 can each be associated with an ASIC 310. Alternatively, the first sensor pixel 302 and the second sensor pixel 304 can be associated with a corresponding ASIC 310. The ASIC 310 can be fabricated in the dielectric layer 204 (e.g., with the temperature sensor 208, the heating element 210a-b and the heat transfer layer 212). Both the dielectric layer 204 and the ASIC 310 can be deposited or formed on the CMOS substrate layer 202a. The ASIC 310 can be mechanically coupled to the CMOS substrate layer 202a. It is to be appreciated that the ASIC 310 can comprise one or more ASIC devices. The ASIC 310 can be configured for controlling heating of the heating element 210a-b, evaluating temperature associated with the ink material 216, determining concentrations of chemicals associated with the ink material 216, etc. In an implementation, the ASIC 310 can include integrated circuitry configured to supply an electrical current to the heating element 210a-b (e.g., so that heating element 210a-b can generate an amount of heat based on the electrical current supplied by the ASIC 310). For example, the ASIC 310 can be a heater control circuit. In another implementation, the ASIC 310 can include integrated circuitry configured to control an operational temperature of the heating element 210a-b. In yet another implementation, the ASIC 310 can include integrated circuitry configured to measure changes associated with the ink material 216 (e.g., measure electrical resistance of the ink material 216, etc). For example, the ASIC 310 can be electrically coupled to the gas-sensing contacts 214a-b.

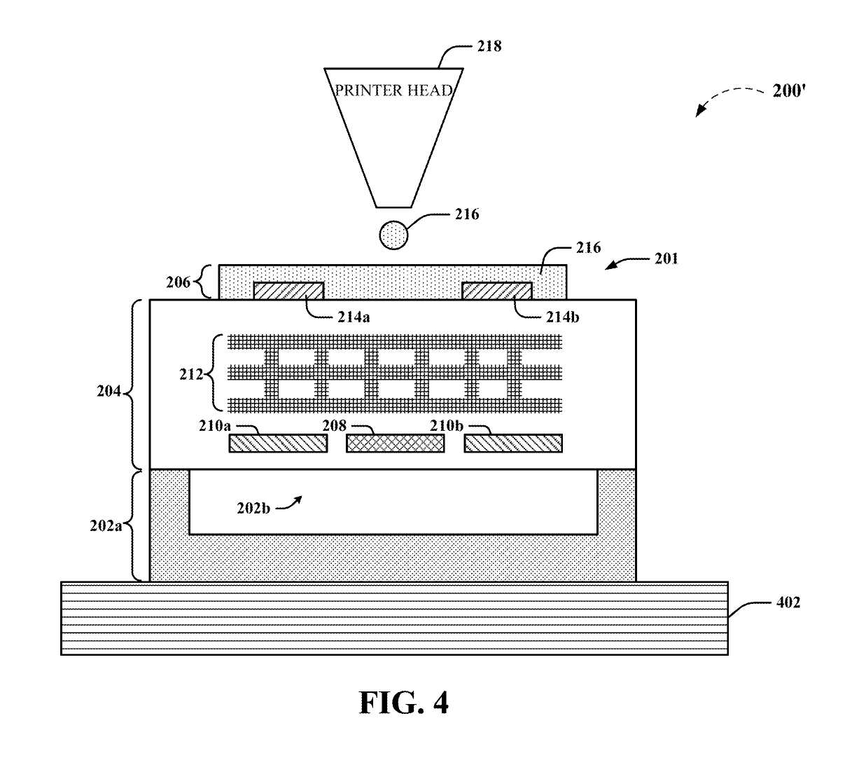

FIG. 4 depicts a system 200′ that includes the gas sensor device 201, according to various non-limiting aspects of the subject disclosure. FIG. 4 depicts a cross-sectional view of the gas sensor device 201. In an implementation, the gas sensor device 201 can include the CMOS substrate layer 202a, the dielectric layer 204 and the gas sensing layer 206. In an implementation, the CMOS substrate layer 202a can include the cavity 202b. The dielectric layer 204 can include the temperature sensor 208, the heating element 210a-b and the heat transfer layer 212. The gas sensing layer 206 can include the set of gas-sensing contacts 214a-b and the ink material 216. Additionally, the system 200′ can include a metal layer 402. The ink material 216 can be printed onto the gas sensor device 201 via the printer head 218. During the printing of the ink material 216 onto the gas sensor device 201 via the printer head 218, the metal layer 402 can be heated and/or heat can be applied to the gas sensor device 201. For example, the metal layer 402 can be heated between 20° C. and 450° C. Therefore, heat from the metal layer 402 can be transferred to the gas sensor device 201 (e.g., to the CMOS substrate layer 202a of the gas sensor device 201). In one example, the metal layer 402 can be a heated chunk of metal. In an implementation, heat can be applied to the metal layer 402 before the ink material 216 is printed onto the gas sensor device 201 via the printer head 218. Additionally or alternatively, heat can be applied to the metal layer 402 of the gas sensor device 201 during printing of the ink material 216 via the printer head 218.

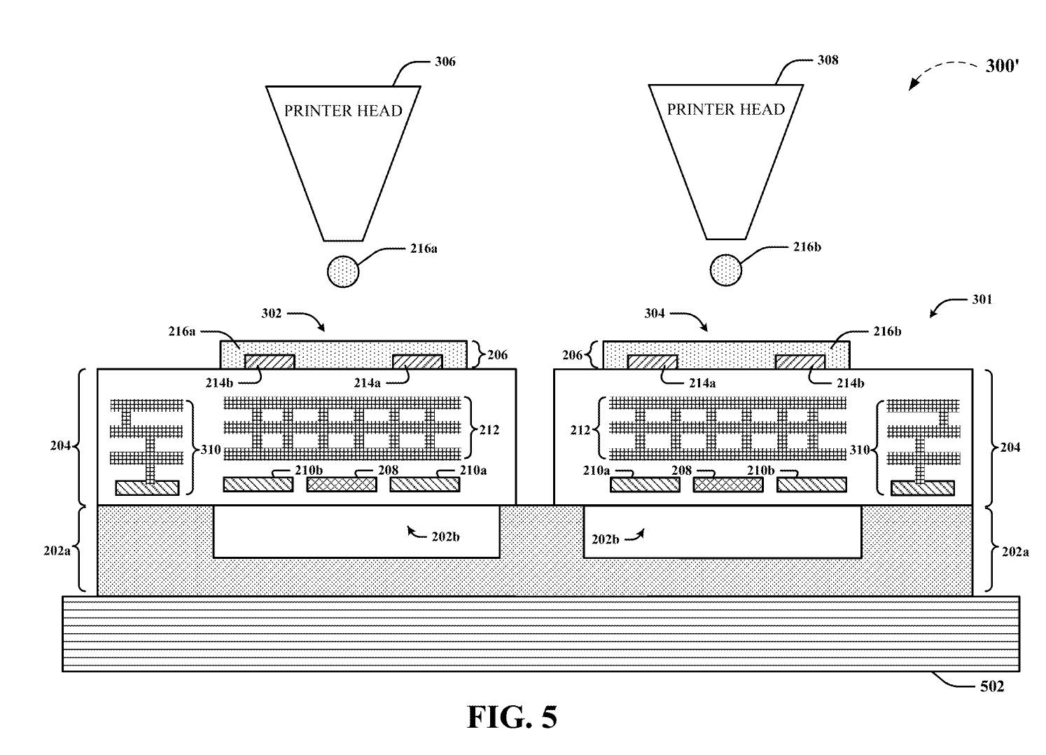

FIG. 5 depicts a system 300′ that includes the gas sensor device 301, according to various non-limiting aspects of the subject disclosure. FIG. 3 depicts a cross-sectional view of the gas sensor device 301. The gas sensor device 301 includes the CMOS substrate layer 202a, the first sensor pixel 302 and the second sensor pixel 304. In an implementation, the CMOS substrate layer 202a can include more than one cavity 202b (e.g., two cavities 202b). The first sensor pixel 302 includes the dielectric layer 204 and the gas sensing layer 206. The dielectric layer 204 of the first sensor pixel 302 can include the temperature sensor 208, the heating element 210a-b and the heat transfer layer 212. Furthermore, the gas sensing layer 206 of the first sensor pixel 302 can include the set of gas-sensing contacts 214a-b and ink material 216a. The second sensor pixel 304 includes the dielectric layer 204 and the gas sensing layer 206. The dielectric layer 204 of the second sensor pixel 304 can include the temperature sensor 208, the heating element 210a-b and the heat transfer layer 212. Furthermore, the gas sensing layer 206 of the second sensor pixel 304 can include the set of gas-sensing contacts 214a-b and ink material 216b. Furthermore, in certain implementation, the first sensor pixel 302 and/or the second sensor pixel 304 can be associated with the ASIC 310. Additionally, the system 300′ can include a metal layer 502. The ink material 216a can be printed onto the gas sensor device 301 via the printer head 306. Furthermore, the ink material 216b can be printed onto the gas sensor device 301 via the printer head 308. During the printing of the ink material 216a onto the gas sensor device 301 via the printer head 306 and/or during the printing of the ink material 216b onto the gas sensor device 301 via the printer head 308, the metal layer 502 can be heated and/or heat can be applied to the gas sensor device 301. For example, the metal layer 502 can be heated between 20° C. and 450° C. Therefore, heat from the metal layer 502 can be transferred to the gas sensor device 301 (e.g., to the CMOS substrate layer 202a of the gas sensor device 301). In one example, the metal layer 502 can be a heated chunk of metal. In an implementation, heat can be applied to the metal layer 502 before the ink material 216a is printed onto the gas sensor device 301 via the printer head 306 and/or before the ink material 216b is printed onto the gas sensor device 301 via the printer head 308. Additionally or alternatively, heat can be applied to the metal layer 502 of the gas sensor device 301 during printing of the ink material 216a via the printer head 306 and/or during printing of the ink material 216b via the printer head 308.

While various embodiments for a gas sensor device according to aspects of the subject disclosure have been described herein for purposes of illustration, and not limitation, it can be appreciated that the subject disclosure is not so limited. Various implementations can be applied to other ink materials, other devices and/or other gas sensing applications, without departing from the subject matter described herein. Furthermore, various exemplary implementations of systems as described herein can additionally, or alternatively, include other features, functionalities and/or components and so on.

In view of the subject matter described supra, methods that can be implemented in accordance with the subject disclosure will be better appreciated with reference to the flowcharts of FIGS. 1 and 6-8. While for purposes of simplicity of explanation, the methods are shown and described as a series of blocks, it is to be understood and appreciated that such illustrations or corresponding descriptions are not limited by the order of the blocks, as some blocks may occur in different orders and/or concurrently with other blocks from what is depicted and described herein. Any non-sequential, or branched, flow illustrated via a flowchart should be understood to indicate that various other branches, flow paths, and orders of the blocks, can be implemented which achieve the same or a similar result. Moreover, not all illustrated blocks may be required to implement the methods described hereinafter.

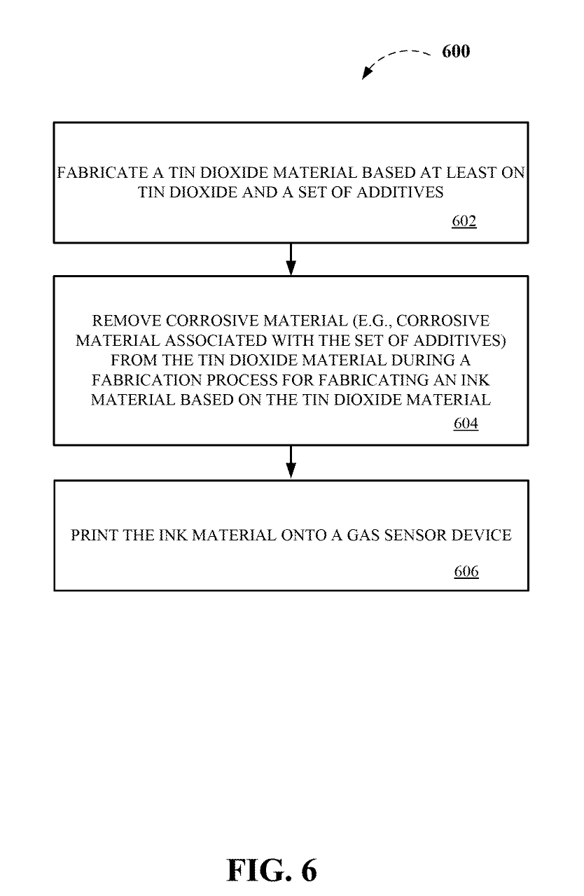

FIG. 6 depicts an exemplary flowchart of a non-limiting method 600 for fabricating ink material (e.g., gas sensing material, chemical sensing material, etc.) for a gas sensor, according to various non-limiting aspects of the subject disclosure. In an aspect, the method 600 can be associated with the system 200, the system 300, the system 200′, the gas system 300′, the gas sensor device 201 and/or the gas sensor device 301. Initially, at 602, a tin dioxide material is fabricated based at least on tin dioxide and a set of additives. The set of additives can include metal chloride, metal acetate and/or ammonium hydroxide. For example, the metal chloride can be associated with platinum, lead, titanium, copper, zinc, lanthanide, iron, gold and/or another type of metal. Additionally or alternatively, the metal acetate can be associated with platinum, lead, titanium, copper, zinc, lanthanide, iron, gold and/or another type of metal. In an aspect, the fabricating the tin dioxide material can include adding metal chloride and metal acetate to a mixture comprising tin dioxide and ammonium hydroxide. For example, a tin dioxide powder can be mixed with ammonium hydroxide, and then metal chloride and metal acetate can be added to the mixture of tin dioxide powder and ammonium hydroxide.

At 604, corrosive material (e.g., corrosive material associated with the set of additives) is removed from the tin dioxide material during a fabrication process for fabricating an ink material based on the tin dioxide material. For example, chloride ions and/or hydroxide ions can be removed from the tin dioxide material via centrifugation (e.g., a process associated with a centrifuge), washing with water (e.g., deionized water) and/or a heat treatment when fabricating an ink material based on the tin dioxide material. In an aspect, the fabricating the ink material includes generating a precipitate substance by adding an organic solvent to the tin dioxide material, generating a slurry mixture from the precipitate substance by performing a centrifugation process and by adding water to the precipitate substance, generating a dry powder material by heating the slurry mixture via the heat treating process, and/or generating the ink material by suspending the dry powder material in a surfactant substance.

At 606, the ink material is printed onto a gas sensor device. The ink material can be a gas-sensing material (e.g., a chemical-sensing material). Furthermore, the ink material can be a printable ink (e.g., a stable suspension ink) for printing onto the gas sensor device. The ink material 216a can comprise at least tin dioxide (e.g., without the corrosive material). In one example, the gas sensor device can be a metal oxide semiconductor gas sensor.

In certain implementations, the method 600 can further include fabricating other tin dioxide material based at least on the tin dioxide and another set of additives that is different than the set of additives, removing corrosive material from the other tin dioxide material during another fabrication process for fabricating another ink material based on the other tin dioxide material, and/or printing the other ink material on another pixel of the gas sensor device. In an aspect, the fabricating the other ink material can include generating a precipitate substance by adding an organic solvent to the other tin dioxide material, generating a slurry mixture from the precipitate substance by performing another centrifugation process and by adding water to the precipitate substance, generating a dry powder material by heating the slurry mixture via another heat treating process, and/or generating the other ink material by suspending the dry powder material in a surfactant substance.

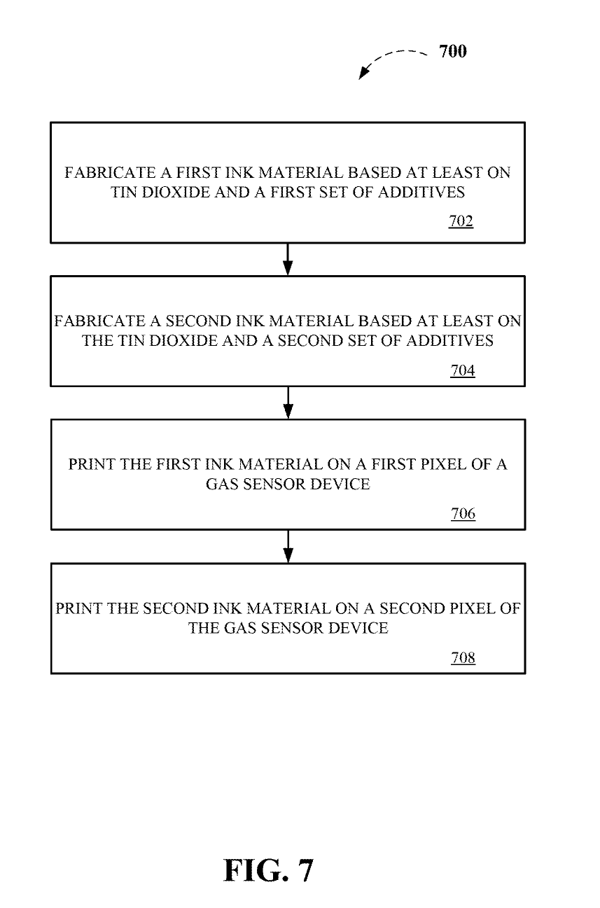

FIG. 7 depicts another exemplary flowchart of a non-limiting method 700 for fabricating ink material (e.g., gas sensing material, chemical sensing material, etc.) for a gas sensor, according to various non-limiting aspects of the subject disclosure. In an aspect, the method 700 can be associated with the system 200, the system 300, the system 200′, the gas system 300′, the gas sensor device 201 and/or the gas sensor device 301. Initially, at 702, a first ink material is fabricated based at least on tin dioxide and a first set of additives. The first set of additives can include metal chloride, metal acetate and/or ammonium hydroxide. For example, the metal chloride of the first set of additives can be associated with platinum, lead, titanium, copper, zinc, lanthanide, iron, gold and/or another type of metal. Additionally or alternatively, the metal acetate of the first set of additives can be associated with platinum, lead, titanium, copper, zinc, lanthanide, iron, gold and/or another type of metal. In an aspect, a tin dioxide powder can be mixed with ammonium hydroxide, and then metal chloride and metal acetate can be added to the mixture of tin dioxide powder and ammonium hydroxide. Next, an organic solvent (e.g., ethanol, ethylene glycol) can be added and/or the stirring can be performed to create precipitates. Then, centrifugation can be performed and/or the precipitates can be washed with water (e.g., deionized water) to obtain a slurry mixture. The slurry mixture can then be heat treated (e.g., between 350° C. and 600° C.) to obtain a dry powder material. The dry powder material can then be suspended in a surfactant to obtain the first ink material. The first ink material can be a gas-sensing material (e.g., a chemical-sensing material). Furthermore, the first ink material can be a printable ink (e.g., a stable suspension ink for printing).

At 704, a second ink material is fabricated based at least on the tin dioxide and a second set of additives. The second set of additives can include metal chloride, metal acetate and/or ammonium hydroxide. For example, the metal chloride of the second set of additives can be associated with platinum, lead, titanium, copper, zinc, lanthanide, iron, gold and/or another type of metal. Additionally or alternatively, the metal acetate of the second set of additives can be associated with platinum, lead, titanium, copper, zinc, lanthanide, iron, gold and/or another type of metal. The second set of additives for the second ink material can be different than the first set of additives for the first ink material. For example, the metal chloride and/or the metal acetate associated with the first set of additives for the first ink material can be different than the metal chloride and/or the metal acetate associated with the second set of additives for the second ink material. In one example, the first set of additives for the first ink material can comprise a different amount of metals (e.g., different amounts of platinum, lead, titanium, copper, zinc, lanthanide, iron, gold and/or other metal) than the second set of additives for the second ink material. In an aspect, a tin dioxide powder can be mixed with ammonium hydroxide, and then metal chloride and metal acetate can be added to the mixture of tin dioxide powder and ammonium hydroxide. Next, an organic solvent (e.g., ethanol, ethylene glycol) can be added and/or the stirring can be performed to create precipitates. Then, centrifugation can be performed and/or the precipitates can be washed with water (e.g., deionized water) to obtain a slurry mixture. The slurry mixture can then be heat treated (e.g., between 350° C. and 600° C.) to obtain a dry powder material. The dry powder material can then be suspended in a surfactant to obtain the second ink material. The second ink material can be a gas-sensing material (e.g., a chemical-sensing material). Furthermore, the second ink material can be a printable ink (e.g., a stable suspension ink for printing).

At 708, the first ink material is printed on a first pixel of a gas sensor device. For example, a first printer head can print the first ink material onto a first sensor pixel implemented on a gas sensor (e.g., a gas sensor substrate). At 710, the second ink material is printed on a second pixel of the gas sensor device. For example, a second printer head can print the second ink material onto a first sensor pixel implemented on a gas sensor (e.g., a gas sensor substrate). As such, the gas sensor can be a multi-pixel gas sensing platform that can sense multiple gases.

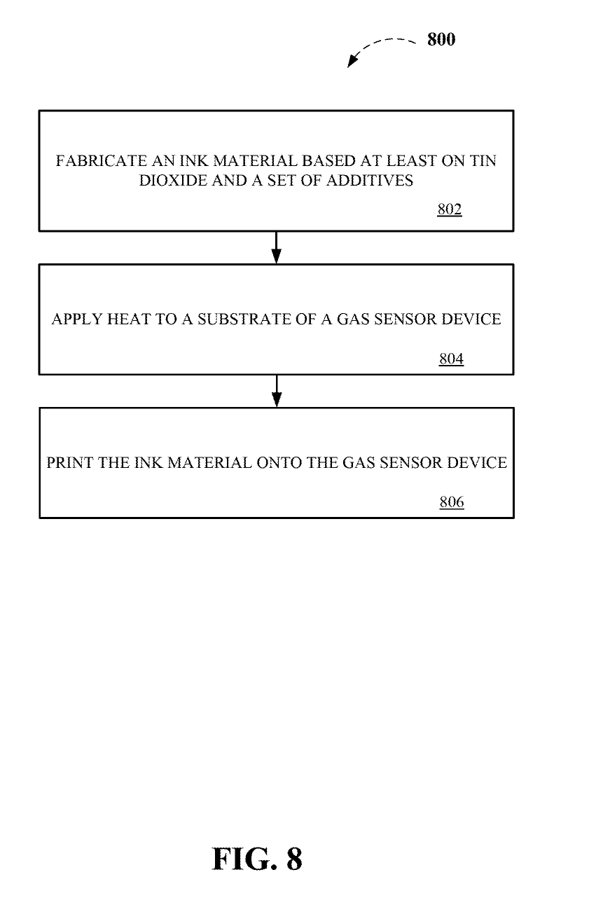

FIG. 8 depicts another exemplary flowchart of a non-limiting method 800 for fabricating ink material (e.g., gas sensing material, chemical sensing material, etc.) for a gas sensor, according to various non-limiting aspects of the subject disclosure. In an aspect, the method 800 can be associated with the system 200, the system 300, the system 200′, the gas system 300′, the gas sensor device 201 and/or the gas sensor device 301. Initially, at 802, an ink material is fabricated based at least on tin dioxide and a set of additives. For example, a tin dioxide powder can be mixed with ammonium hydroxide, and then metal chloride and metal acetate can be added to the mixture of tin dioxide powder and ammonium hydroxide. Next, an organic solvent (e.g., ethanol, ethylene glycol) can be added and/or the stirring can be performed to create precipitates. Then, centrifugation can be performed and/or the precipitates can be washed with water (e.g., deionized water) to obtain a slurry mixture. The slurry mixture can then be heat treated (e.g., between 350° C. and 600° C.) to obtain a dry powder material. The dry powder material can then be suspended in a surfactant to obtain the ink material. The ink material can be a gas-sensing material (e.g., a chemical-sensing material). Furthermore, the ink material can be a printable ink (e.g., a stable suspension ink) for printing.

At 804, heat is applied to a substrate of a gas sensor device. For example, a metal layer thermally coupled to the substrate of the gas sensor can be heated. Therefore, heat from the metal layer can be transferred to the substrate of the gas sensor device. The metal layer can be, for example, a heated metal chunk. In one example, the metal layer can be heated between 20° C. and 450° C.

At 806, the ink material is printed onto the gas sensor device. For example, a printer head can print the ink material onto a sensor pixel implemented on the gas sensor device (e.g., on the substrate of the gas sensor device). In an implementation, the heat can be applied to the substrate of the gas sensor device before the ink material is printed onto the gas sensor device. Additionally or alternatively, the heat can be applied to the substrate of the gas sensor device during the printing of the ink material onto the gas sensor device.

It is to be appreciated that various exemplary implementations of exemplary methods 100, 600, 700 and 800 as described can additionally, or alternatively, include other process steps for fabricating ink material for a gas sensor and/or printing the ink material onto the gas sensor, as further detailed herein, for example, regarding FIGS. 2-5.

What has been described above includes examples of the embodiments of the subject disclosure. It is, of course, not possible to describe every conceivable combination of configurations, components, and/or methods for purposes of describing the claimed subject matter, but it is to be appreciated that many further combinations and permutations of the various embodiments are possible. Accordingly, the claimed subject matter is intended to embrace all such alterations, modifications, and variations that fall within the spirit and scope of the appended claims. While specific embodiments and examples are described in subject disclosure for illustrative purposes, various modifications are possible that are considered within the scope of such embodiments and examples, as those skilled in the relevant art can recognize.

In addition, the words “example” or “exemplary” is used herein to mean serving as an example, instance, or illustration. Any aspect or design described herein as “exemplary” is not necessarily to be construed as preferred or advantageous over other aspects or designs. Rather, use of the word, “exemplary,” is intended to present concepts in a concrete fashion. As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

In addition, while an aspect may have been disclosed with respect to only one of several embodiments, such feature may be combined with one or more other features of the other embodiments as may be desired and advantageous for any given or particular application. Furthermore, to the extent that the terms “includes,” “including,” “has,” “contains,” variants thereof, and other similar words are used in either the detailed description or the claims, these terms are intended to be inclusive in a manner similar to the term “comprising” as an open transition word without precluding any additional or other elements.

What is claimed is:

1. A method, comprising:
generating a tin dioxide material by adding metal chloride and metal acetate to a mixture comprising tin dioxide and ammonium hydroxide;
generating a precipitate substance by adding an organic solvent to the tin dioxide material;
generating a slurry mixture from the precipitate substance by performing a centrifugation process and by adding water to the precipitate substance;
generating a dry powder material by heating the slurry mixture via a heat treating process;
generating an ink material by suspending the dry powder material in a surfactant substance; and
printing the ink material onto a gas sensor.

2. The method of claim 1, wherein the metal chloride comprises platinum, lead, titanium, copper, zinc, lanthanide, iron or gold.

3. The method of claim 1, wherein the adding the organic solvent to the tin dioxide material comprises stirring the organic solvent with the tin dioxide material.

4. The method of claim 1, wherein the adding the water to the precipitate substance comprises adding deionized water to the precipitate substance.

5. The method of claim 1, wherein the heating the slurry mixture comprises heat treating the slurry mixture between 350° C. and 600° C.

6. The method of claim 1, wherein the generating the ink material comprises generating a gas sensing material for the gas sensor.

7. The method of claim 1, wherein the printing the ink material onto the gas sensor comprises sintering the ink material at a temperature lower than 450° C.

8. The method of claim 1, wherein the printing the ink material onto the gas sensor comprises heating a substrate of the gas sensor before the printing.

9. The method of claim 1, wherein the printing the ink material onto the gas sensor comprises heating a substrate of the gas sensor during the printing.

10. A method, comprising:
fabricating a tin dioxide material based at least on tin dioxide and a set of additives;
removing corrosive material from the tin dioxide material during a fabrication process for fabricating an ink material based on the tin dioxide material, comprising performing a centrifugation process and performing a heat treating process; and
printing the ink material on a pixel of a gas sensor device.

11. The method of claim 10, wherein the fabricating the tin dioxide material comprises adding metal chloride and metal acetate to a mixture comprising tin dioxide and ammonium hydroxide.

12. The method of claim 10, wherein the fabricating the ink material comprises:
generating a precipitate substance by adding an organic solvent to the tin dioxide material, and
generating a slurry mixture from the precipitate substance by performing the centrifugation process and by adding water to the precipitate substance.

13. The method of claim 12, wherein the fabricating the ink material comprises:
generating a dry powder material by heating the slurry mixture via the heat treating process, and
generating the ink material by suspending the dry powder material in a surfactant substance.

14. The method of claim 10, further comprising:
fabricating other tin dioxide material based at least on the tin dioxide and another set of additives that is different than the set of additives.

15. The method of claim 14, further comprising:
removing corrosive material from the other tin dioxide material during another fabrication process for fabricating another ink material based on the other tin dioxide material.

16. The method of claim 15, further comprising:
printing the other ink material on another pixel of the gas sensor device.

17. The method of claim 15, wherein the fabricating the other ink material comprises:
generating a precipitate substance by adding an organic solvent to the other tin dioxide material, and
generating a slurry mixture from the precipitate substance by performing another centrifugation process and by adding water to the precipitate substance.

18. The method of claim 12, wherein the fabricating the other ink material comprises:
generating a dry powder material by heating the slurry mixture via another heat treating process, and
generating the other ink material by suspending the dry powder material in a surfactant substance.

19. A method, comprising:
fabricating a tin dioxide material based at least on tin dioxide and a set of additives;
removing corrosive material from the tin dioxide material during a fabrication process for fabricating an ink material based on the tin dioxide material; and
printing the ink material on a pixel of a gas sensor device, comprising heating a substrate of the gas sensor device.

20. The method of claim 19, wherein the heating the substrate comprises heating a metal layer thermally coupled to the substrate of the gas sensor device.
